(19) United States
(12) Patent Application Publication
(10) Pub. No.: US 2023/0122156 A1
(43) Pub. Date: Apr. 20, 2023

(54) AI-BASED TOOL FOR SCREENING SLEEP APNEA

(71) Applicant: West Virginia University Board of Governors on Behalf of West Virginia University, Morgantown, WV (US)

(72) Inventors: SUNIL SHARMA, MORGANTOWN, WV (US); NAVEENA YANAMALA, MORGANTOWN, WV (US)

(21) Appl. No.: 18/046,632

(22) Filed: Oct. 14, 2022

Related U.S. Application Data
(60) Provisional application No. 63/256,251, filed on Oct. 15, 2021.

(51) Int. Cl. A61B 5/00; A61B 5/1455

(52) U.S. Cl. CPC A61B 5/4818; A61B 5/002; A61B 5/742; A61B 5/4842; A61B 5/7267; A61B 5/7275; A61B 5/7475; A61B 5/14551

(57) ABSTRACT

Systems and methods for sleep apnea and nocturnal hypoxia detection are described. In one non-limiting example, a pulse oximetry device can include a sensor and a computing device. The computing device can be attached to a patient for a sleep apnea diagnosis. The computing device can be configured to generate pulse oximetry data by measuring pulse oximetry of a patient with the sensor. Multiple apnea indicators can be identified from the pulse oximetry data and from information associated with the patient. The multiple sleep apnea indicators can be provided to a machine learning model trained for sleep apnea prediction. A sleep apnea classification can be determined from the machine learning model.

### CROSS REFERENCE TO RELATED PATENT APPLICATIONS

This application claims the benefit of and priority to U.S. Provisional Pat. Application 63/256,251, entitled “AI-Based Tool for Screening Sleep Apnea,” and filed on Oct. 15, 2021, which is incorporated herein by reference in its entirety.

### BACKGROUND

Sleep apnea is a highly prevalent condition that is under-diagnosed. Clinical screening is not accurate and confirmation tests costs hundreds of dollars. People with untreated sleep apnea are at an increased risk of cardiovascular complications including heart failure, which is a common cause of hospital admissions.

### SUMMARY

Embodiments of the present disclosure are related to improved sleep apnea and nocturnal hypoxia detection systems and methods that provide a better experience for a user wearing a pulse oximetry device during a sleep apnea diagnosis.

According one embodiment, among others, a system is provided comprising a computing device that comprises a processor and a memory; and machine-readable instructions stored in the memory that, when executed by the processor, cause the computing device to at least: receive patient data that comprises at least one of demographic or clinical information associated with a patient; receive pulse oximetry data that was measured by a pulse oximetry device worn by the patient; generate oximetry characteristics based at least in part on the pulse oximetry data and a plurality of parameter thresholds, the oximetry characteristics comprising a plurality processed parameters; select a plurality of sleep apnea indicators based at least in part on a sleep apnea profile associated with a patient, the patient data, and the oximetry characteristics; and training a machine learning model for a sleep apnea prediction based at least in part on the plurality of sleep apnea indicators, wherein the sleep apnea prediction representing an apnea classification for the patient.

According one embodiment, among others, a portable system is provided comprising a sensor; a computing device that comprises a processor and a memory, the computing device being attached to a patient for a sleep apnea diagnosis; and machine-readable instructions stored in the memory that, when executed by the processor, cause the computing device to at least: generate pulse oximetry data by measuring pulse oximetry of a patient with the sensor at a sampling rate of less than three seconds; identify a plurality of sleep apnea indicators from the pulse oximetry data and from patient information associated with the patient; provide the plurality of sleep apnea indicators to a machine learning model trained for sleep apnea prediction; and receive a sleep apnea classification from the machine learning model.

According one embodiment, among others, a method is provided comprising the steps of generating, by a portable device that includes a sensor, pulse oximetry data by measuring pulse oximetry of a patient with the sensor at a sampling rate of less than three seconds; identifying, by the portable device, a plurality of sleep apnea indicators from the pulse oximetry data and from patient information associated with the patient; providing, by the portable device, the plurality of sleep apnea indicators to a machine learning model trained for sleep apnea prediction; and receiving, by the portable device, a sleep apnea classification from the machine learning model.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims.

In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the following drawings. The components in the drawings are not necessarily to scale, with emphasis instead being placed upon clearly illustrating the principles of the disclosure. Moreover, in the drawings, like reference numerals designate corresponding parts throughout the several views.

FIG. 1 is a drawing of a networked environment according to various embodiments of the present disclosure.

FIG. 2 is a flowchart illustrating one example of functionality implemented as portions of apnea machine learning service executed in a computing environment in the networked environment of FIG. 1 according to various embodiments of the present disclosure.

FIG. 3 is a flowchart illustrating one example of functionality implemented as portions of oximetry application executed in a pulse oximetry device in the networked environment of FIG. 1 according to various embodiments of the present disclosure.

FIG. 4 is a pictorial diagram of an example user interface rendered by a client device in the networked environment of FIG. 1 according to various embodiments of the present disclosure.

FIG. 5 is table of data for evaluating machine learning model trained for identify sleep apnea according to various embodiments of the present disclosure.

### DETAILED DESCRIPTION

The embodiments of the present disclosure relate to improved sleep apnea and nocturnal hypoxia detection systems and methods that provide a better experience for a user wearing a pulse oximetry device during a sleep apnea diagnosis. Additionally, the embodiments provide an improved accuracy for predicting whether the user has sleep apnea in real-time (or near-real time) based on a pulse oximetry device in a home setting. The embodiments can be operated and understood by the patient without further analysis and guidance from a medical professional.

Various embodiments of the present disclosure relate to systems and methods that use an artificial intelligence based platform for analyzing data from a pulse oximeter device and patient demographic/clinical information to accurately predict not only the disease state but its severity and other related hypoxia causing conditions. The embodiments can generate deep learning and machine learning models that demonstrate higher overall sensitivity, specificity and accuracy compared to previous studies that detect such ailments. Apart from sleep apnea, this can detect the presence and severity of oxygen loss during sleep which may be an early sign of lung or heart conditions including but not limited to Chronic Obstructive Pulmonary Disease (COPD), Congestive Heart Failure (CHF), and infectious etiologies. The embodiments are user-friendly and can be used in a home setting as a prescreening tool to detect sleep apnea, providing guidance for the further need of consultation from a medical professional. Since sleep apnea is highly underdiagnosed and may lead to fatal outcomes if not recognized, it is important that sleep apnea gets detected and managed early. As such, the embodiments can drastically improve the detection and subsequent diagnosis of sleep apnea by providing greater access to people silently suffering from this disease. Existing technology is costly, not user friendly, and requires an order from a physician which adds to the cost and delay in detecting the disorder. Additionally, existing diagnoses of sleep apnea require the use of more than just a pulse oximetry device. As such, the embodiments are directed to improved systems and methods for detecting sleep apnea and hypoxia-related conditions for at least the reason that a pulse oximetry device is the only diagnosis device being worn by the patient. In the following discussion, a general description of the system and its components is provided, followed by a discussion of the operation of the same.

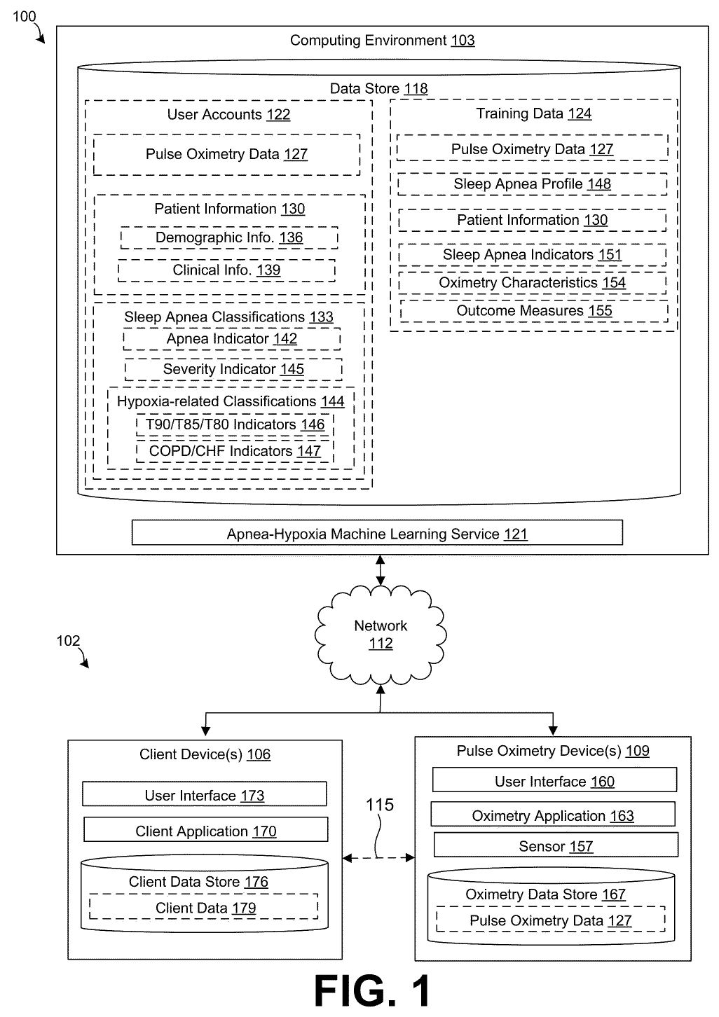

With reference to FIG. 1, shown is a networked environment 100 according to various embodiments. The networked environment 100 includes a computing environment 103, and a sleep apnea detection system 102 (e.g., a client device 106, a pulse oximetry device 109), which are in data communication with each other via a network 112. The network 112 includes, for example, the Internet, intranets, extranets, wide area networks (WANs), local area networks (LANs), wired networks, wireless networks, or other suitable networks, etc., or any combination of two or more such networks. For example, such networks may comprise satellite networks, cable networks, Ethernet networks, and other types of networks. The client device 106 and the pulse oximetry device 109 can also be in data communication via wireless communication 115. The wireless communication 115 can include Bluetooth protocols, Zigbee protocols, Wireless USB, Near Field Communication (NFC), Z-Wave, and other short-range wireless communication protocols.

The computing environment 103 may comprise, for example, a server computer, Advanced RISC machine (ARM) chips containing SOC or any other system providing computing capability. Alternatively, the computing environment 103 may employ a plurality of computing devices that may be arranged, for example, in one or more server banks or computer banks or other arrangements. Such computing devices may be located in a single installation or may be distributed among many different geographical locations. For example, the computing environment 103 may include a plurality of computing devices that together may comprise a hosted computing resource, a grid computing resource an edge computing distributed network, and/or any other distributed computing arrangement. In some cases, the computing environment 103 may correspond to an elastic computing resource where the allotted capacity of processing, network, storage, or other computing-related resources may vary over time.

Various applications and/or other functionality may be executed in the computing environment 103 according to various embodiments. Also, various data is stored in a data store 118 that is accessible to the computing environment 103. The data store 118 may be representative of a plurality of data stores 118 as can be appreciated. The data stored in the data store 118, for example, is associated with the operation of the various applications and/or functional entities described below.

The components executed on the computing environment 103, for example, include an apnea and hypoxia machine learning service 121, deep learning/machine learning services for hypoxia related conditions and other applications, services, processes, systems, engines, or functionality not discussed in detail herein. The apnea and hypoxia machine learning service 121 121 is executed to train one or more machine learning models for predicting sleep apnea classifications. The apnea and hypoxia machine learning service 121 can also deploy one or more trained machine learning models to provide client devices 106 or pulse oximetry devices with sleep apnea prediction classifications. In some non-limiting examples, the apnea and hypoxia machine learning service 121 can executed on an edge computing device. In some implementations, edge computing can refer a distributed computing network that may include one or more edge computing nodes (e.g., Internet of Things (IoT) nodes, IoT gateways).

The data stored in the data store 118 includes, for example, user accounts 122 and training data 124, and potentially other data. The user account 122 can represent an account or a profile that is unique for a user. The user account 122 can include a pulse oximetry data 127, patient information 130, sleep apnea classifications 133, and other suitable information. The pulse oximetry data 127 can represent oximetry measurement data provided by the pulse oximetry device 109. The patient information 130 can represent data associated with the patient that is being diagnosed for sleep apnea. The patient information 130 can include demographic information 136 and clinical information 139. The demographic information 136 can include an age of the patient, a race, an ethnicity, gender, and other demographic information. The clinical information 139 can include a body mass index, a weight, a height, blood pressure measurements, history of hypertension and other clinical information, which can reside within the controlled environment of the user or patient.

The sleep apnea and hypoxia classification 133 can represent one or more classifications that have been determined by the apnea and hypoxia machine learning service 121, machine learning or deep learning models, the client application, and/or the oximetry application. The sleep apnea classification 133 can include an apnea indicator 142 for indicating whether the patient has sleep apnea (e.g., yes or no). The sleep apnea and hypoxia classification 133 can also include a severity indicator 145, such as low, medium, high, or other severity indicators. Some other severity indicators can include hypoxia-related classifications 144 can include threshold indicators 146 T90 (time spent below 90%), T85 (time spent below 85%), T80 (time spent below 80%) or NADIR of oxygen level. Hypoxia-related classifications 144 can also include COPD/CHF indicators 147. The severity indicator 145 may have a threshold associated with each severity level (e.g., low, medium, or high).

The training data 124 can represent data used for training one or more machine learning model by apnea and hypoxia machine learning service 121. The training data 124 can include the pulse oximetry data 127, a sleep apnea profile 148, the patient information 130, sleep apnea indicators 151, oximetry characteristics 154, outcome measures 155, and other suitable data.

The sleep apnea profile 148 can represent a profile of oximetry data of an identified sleep apnea diagnosis from a prior sleep apnea study. The sleep apnea profile 148 can be used for comparison purposes to determine sleep apnea indicators 151. For example, sleep apnea profile 148 can provide one or examples of oxygen related measurements of verified sleep apnea diagnosis.

The patient information 130 and the pulse oximetry data can be used for training a machine learning model. The sleep apnea indicators 151 can be identified from the raw pulse oximetry signals, processed parameters, patient information, and other suitable data. The sleep apnea indicators 151 represent that the most impactful identified data or variables for predicting sleep apnea and its severity level. In some embodiments, the sleep apnea indicators are an oxygen saturation level, a desaturation index (ODI), the lowest oxygen level measured for a night of sleep, and other suitable measurements.

The oximetry characteristics 154 processes data that is generated from the pulse oximetry data 127. As a non-limiting example, the oximetry characteristics 154 can include processed parameters that are generated based on a raw oximetry data signal compared to a threshold. The outcome measures 155 represents mortality, hospital readmission rate and unplanned episodes of care, improved sleep quality indices, and other suitable measures.

The pulse oximetry device 109 is a device that measures oxygen levels or oxygen saturation levels in the blood of a patient. The pulse oximetry device 109 can attach to a body part, such as a finger, a toe, an earlobe and other suitable body locations. In some embodiments, the pulse oximetry device is considered a high resolution pulse oximetry (HRPO) device. Some non-limiting example characteristics of the HRPO can include a signal resolution of 0.1% oxygen saturation (SpO2). The HRPO device can also include an averaging time (e.g., a moving window average) that is shorter traditional pulse oximetry devices. The moving window averages data across time intervals during a test. For example, the HRPO may have an averaging time of three seconds or less. The HRPO device can also have a sampling rate of less than four seconds.

The pulse oximetry device 109 can include a sensor 157 (e.g., a sensor with System on a Chip), a user interface 160, an oximetry application 163, and an oximetry data store 167. The sensor 157 can be used to take oxygen-related measurements. For example, the sensor 157 can include a light detector and a light emitting diode for taking oxygen related measurements. In some embodiments, the pulse oximetry device 109 can be a fitness tracking device, a watch, or other suitable wearable devices. In some embodiments, the pulse oximetry device 109 can be constructed as a portable system, a portable device, and other suitable format factors.

The user interface 160 can display information from the oximetry application 163. In some embodiments, the user interface 160 can display a sleep apnea classification 133. The oximetry application 163 can be executed to take oxygen-related measurements via the sensor 157. In some examples, the oximetry application 163 can interact with the apnea and hypoxia machine learning service 121 to provide sleep apnea classifications 133. The oximetry data store 167 can store pulse oximetry data 127 measured or generated by the pulse oximetry device 109.

The client device 106 is representative of a plurality of client devices that may be coupled to the network 112. The client device 106 may comprise, for example, a processor-based system such as a computer system. Such a computer system may be embodied in the form of a desktop computer, a laptop computer, personal digital assistants, cellular telephones, smartphones, set-top boxes, music players, web pads, tablet computer systems, game consoles, electronic book readers, or other devices with like capability. The client device 106 may include a display. The display may comprise, for example, one or more devices such as liquid crystal display (LCD) displays, gas plasma-based flat panel displays, organic light emitting diode (OLED) displays, electrophoretic ink (E ink) displays, LCD projectors, or other types of display devices, etc.

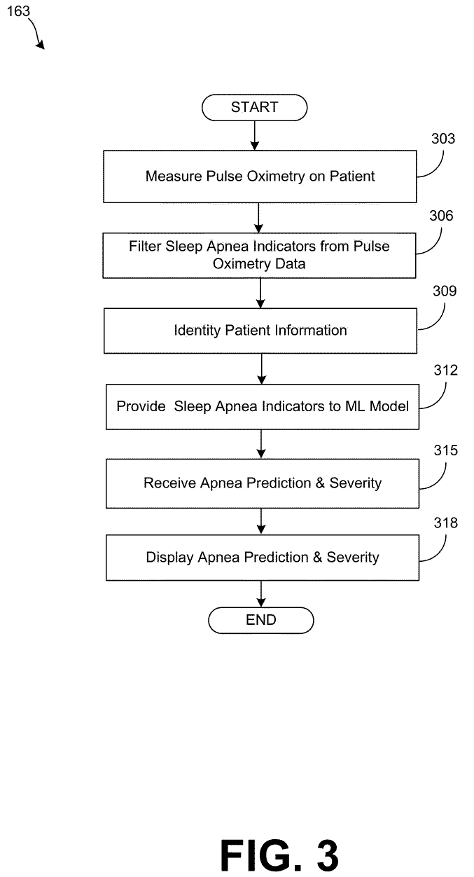

The client device 106 may be configured to execute various applications such as a client application 170 and/or other applications. The client application 170 may be executed in a client device 106, for example, to access network content served up by the computing environment 103 and/or other servers, thereby rendering a user interface 173 on the display. To this end, the client application 170 may comprise, for example, a browser, a dedicated application, etc., and the user interface 173 may comprise a network page, an application screen, etc. The client device 106 may be configured to execute applications beyond the client application 170 such as, for example, email applications, social networking applications, word processors, spreadsheets, and/or other applications. The client application 170 can be executed to communicate with the pulse oximetry device 109 and/or the apnea and hypoxia machine learning service 121. In some embodiments, the client application 170 can generate the user interface 173 to include sleep apnea classifications 133 (e.g., FIG. 3).

The client device 106 can also include a client device store 176 to store client data 179. In some examples, the client data 170 (e.g., smart phone or tablet) can store raw data (e.g., pulse oximetry data 127) from the pulse oximetry device 109. The raw data can be managed and control the patient in order to preserve data privacy and security. The raw data can also be stored and managed at an edge computing device associated with the computing environment or another third party.

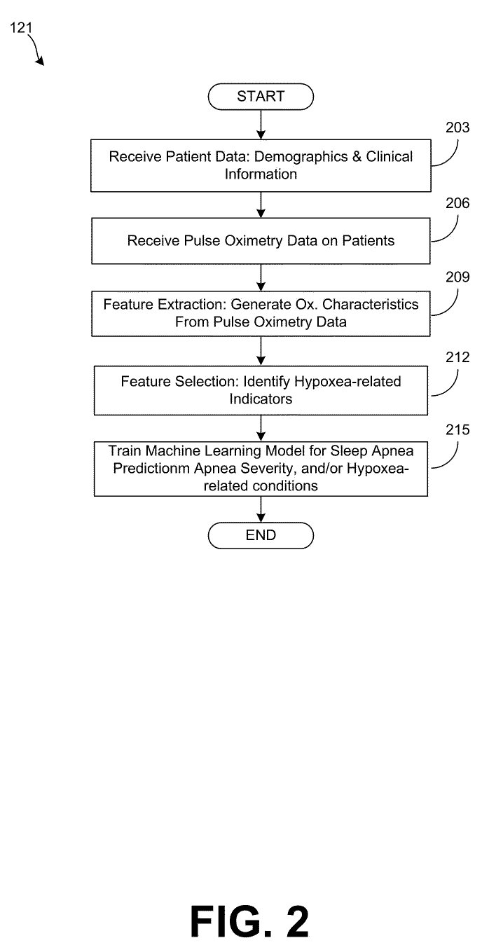

Referring next to FIG. 2, shown is a flowchart that provides one example of the operation of a portion of the apnea and hypoxia machine learning service 121 according to various embodiments. FIG. 2 can be one example of training a machine learning model for predicting sleep apnea classification 133. It is understood that the flowchart of FIG. 2 provides merely an example of the many different types of functional arrangements that may be employed to implement the operation of the portion of the apnea and hypoxia machine learning service 121 as described herein. As an alternative, the flowchart of FIG. 2 may be viewed as depicting an example of elements of a method implemented in the computing environment 103 (FIG. 1) according to one or more embodiments.

Beginning with box 203, the apnea and hypoxia machine learning service 121, which can be deployed on an edge computing device or on the client device 106, can receive demographic information 136 and clinical information 139. The demographic information 136 can include receiving a body mass index and/or the age of the multiple patients.

In box 206, the apnea and hypoxia machine learning service 121 can receive pulse oximetry data 127 that has been generated from pulse oximetry devices 109. The pulse oximetry data 127 can represent raw oxygen-related signals or measurements obtained from the pulse oximetry devices 109. In some embodiments, either the raw oxygen-related signals are extracted from the memory of the pulse oximetry device 109 or the related parameter after pre-processing the data on the sensor device will be pushed as input to the apnea and hypoxia machine learning service.

In box 209, the apnea-hypoxia machine learning service 121 can perform a feature extraction on the pulse oximetry data 127. The apnea and hypoxia machine learning service 121 can process the pulse oximetry data 127 to generate oximetry characteristics or obtain them directly from the sensor device that describe the pulse oximetry data 127. The oximetry characteristics can include processed parameters, such as the lowest detected oxygen level detected, oxygen desaturated index, a number of oxygen readings that dropped below a threshold, and other oxygen related measurements or derived data.

In box 212, the apnea and hypoxia machine learning service 121 can perform feature selection to identify sleep apnea indicators. The sleep apnea indicators can be determined by identifying the most impactful data (e.g., sleep apnea indicators 151) that indicates a sleep apnea or hypoxia-related other conditions classification 133. As such, the sleep apnea and hypoxia indicators can be a subset of data that is selected from the oximetry characteristics, the patient information (e.g. demographic and clinical information), the pulse oximetry data 127, and other suitable data. In some embodiments, the analysis for the sleep apnea indicators can be identified based on a comparison to a sleep apnea profile that has a verified diagnosis by a medical professional (e.g., before and during treatment to ensure that the disease is well managed). In some embodiments, the apnea and hypoxia machine learning service 121 can use a machine learning or deep learning model to compare the sleep apnea and/or hypoxia profiles with the oximetry characteristics, the patient information (e.g., demographic information), the pulse oximetry data 127, and other suitable data. Some non-limiting examples, the sleep apnea indicators 151 can be identified to be age, body mass index, an oxygen saturation level (SpO2), an oxygen desaturation index (ODI), a lowest oxygen measurement detected for the time period, and other suitable factors.

In box 215, the apnea and hypoxia machine learning service 121 can provide the identified sleep apnea indicators to a machine learning model for training, in which the machine learning model continues to improve the precision as a screening tool for detecting sleep apnea, its severity level and/or other hypoxia-related conditions. In some embodiments, the apnea and hypoxia machine learning service 121 can use supervised machine learning or deep learning approaches. Different classification algorithms can be used for training a machine learning for sleep apnea classification. In one non-limiting example, an ensemble machine learning algorithm can be used for classification, such as a Random Forest Model. However, other ensemble machine learning algorithms can be used. Additionally, neural networks and gradient boosting algorithms can also be used as other possible examples. Then, the apnea and hypoxia machine learning service 121 proceeds to the end.

Referring next to FIG. 3, shown is a flowchart that provides one example of the operation of a portion of the oximetry application 163 according to various embodiments. FIG. 3 represents one example of the functionality executed by the oximetry application 163 in a pulse oximetry device 109. It is understood that the flowchart of FIG. 3 provides merely an example of the many different types of functional arrangements that may be employed to implement the operation of the portion of the oximetry application 163 as described herein. As an alternative, the flowchart of FIG. 3 may be viewed as depicting an example of elements of a method implemented in the pulse oximetry device 109 (FIG. 1) according to one or more embodiments. As another alternative embodiment, the flowchart of FIG. 3 may be viewed as depicted a method executed, in part or as a whole, by the client application 170 in the client device 106.

Beginning with box 303, it can be assumed the pulse oximetry device 109 is being worn by a patient for a sleep apnea diagnosis and hypoxia-related conditions. The oximetry application 163 can measure pulse oximetry data 127 from the patient. The pulse oximetry device 109 may collect pulse oximetry data 127 over a night of sleep (e.g., six hours or more) for the patient at home.

In box 306, the oximetry application 163 can filter sleep apnea indicators from the pulse oximetry data 127. In some embodiments, the sleep apnea indicators may be predefined as certain signals to extract from the pulse oximetry data 127 or as certain oximetry characteristics data to generate from the pulse oximetry data 127. For example, the sleep apnea indicators may be identified during a training phase of the machine learning model either on sensor device or on the client devices 106.

In box 309, the oximetry application 163 can identify patient information. In some embodiments, certain demographic or clinical information can be added to the sleep apnea indicators. For example, the body mass index of the patient or an age of the patient can be added to the sleep apnea indicators.

In box 312, the oximetry application 163 can provide the sleep apnea indicators to a trained machine learning model that is deployed to provide sleep apnea classifications 133. The oximetry application 163 can transmit the sleep apnea indicators to the apnea and hypoxia machine learning service 121. In some embodiments, the apnea and hypoxia machine learning service 121 may query a trained machine learning model with the sleep apnea indicators. The trained machine learning model can provide a response that include one or more sleep apnea classifications 133 (e.g., apnea indicator 142 and severity indicator 145).

In box 315, the oximetry application 163 can receive the sleep apnea classification 133. In some embodiments, the sleep apnea classification 133 can be received by the client device 106. In another embodiment, the machine learning model may be executed on a client device 106 or the pulse oximetry device 109. As such, the apnea classifications 133 can be determined locally.

In box 318, the oximetry application 163 can display a use interface 160 can include sleep apnea classifications 133, such as indication of whether the patient has sleep apnea and a level of severity. The use interface 160 can also display oximetry characteristics, pulse oximetry data, and other suitable information.

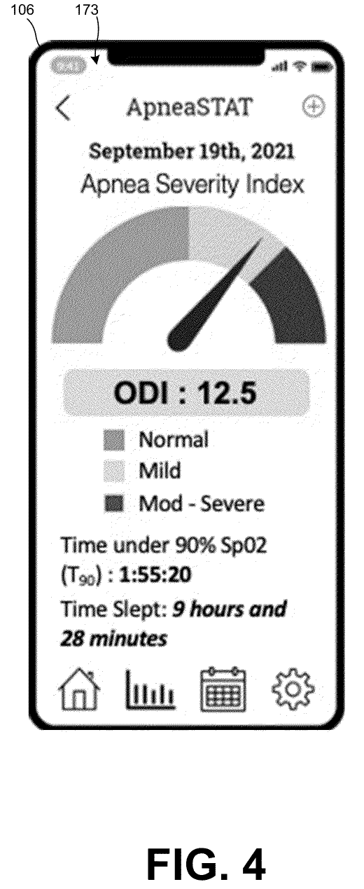

Referring next to FIG. 4, shown is a user interface 173 displayed on the client device 106. The user interface 173 displays a visual apnea gauge with a gauge indicator. The gauge indicator can operate a severity indicator 145 to indicate the level of sleep apnea. The user interface 173 also include a statistics for a period of time (e.g., a night sleep). In the depicted example, the statistics include a desaturation index (ODI), the lowest oxygen measurement for the night, an average level of oxygen, time spent under an oxygen related threshold (e.g., 90%, 85%, 80%), a period of time for which the patient was sleep, and other suitable parameters.

The user interface 173 also include other visual icons for additional functionality, such as a home icon, a graph icon, a calendar icon, and a settings icon. The home icon can be used to display a home user interface. The graph icon can be used to display a user interface that includes one or more graphs of the sleep apnea classifications 133, user account 122 (e.g., pulse oximetry data 127), and other suitable data. The calendar icon can be used for display a calendar user interface, which can include sleep apnea statistics related to a time period. The settings icon can be used to display a user interface for configuring settings for displaying data associated with the user account 122, for configuring settings for the pulse oximetry device 109, and other suitable settings. Other icon and functionality can be included in the user interface 173.

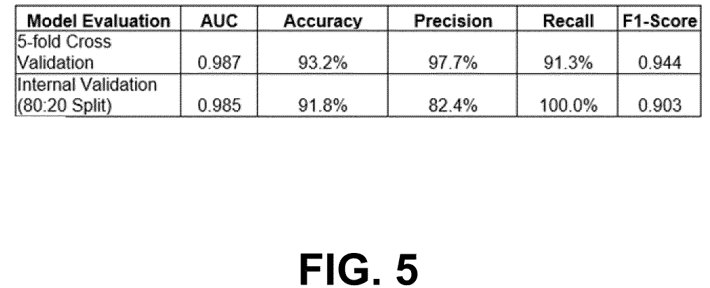

Turning now to FIG. 5, shown is table that includes data captured for evaluating the trained machine learning model at identifying sleep apnea.

Disjunctive language such as the phrase “at least one of X, Y, or Z,” unless specifically stated otherwise, is otherwise understood with the context as used in general to present that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

1. A system, comprising:
a computing device that comprises a processor and a memory; and
machine-readable instructions stored in the memory that, when executed by the processor, cause the computing device to at least:
receive patient data that comprises at least one of demographic or clinical information associated with a patient;
receive pulse oximetry data that was measured by a pulse oximetry device worn by the patient;
generate oximetry characteristics based at least in part on the pulse oximetry data and a plurality of parameter thresholds, the oximetry characteristics comprising a plurality processed parameters;
select a plurality of sleep apnea indicators based at least in part on a sleep apnea profile associated with a patient, the patient data, and the oximetry characteristics; and
training a machine learning model for a sleep apnea prediction based at least in part on the plurality of sleep apnea indicators, wherein the sleep apnea prediction representing an apnea classification for the patient.

2. The system of claim 1, wherein the pulse oximetry device is a high resolution continuous pulse oximetry device that has a signal resolution of at least 0.1% for an oxygen saturation level measurement.

3. The system of claim 1, wherein the plurality of sleep apnea indicators comprises at least one oxygen saturation level, or an oxygen desaturation index.

4. The system of claim 1, wherein the apnea classification is determined based at least in part on a severity threshold.

5. The system of claim 1, wherein the oximetry characteristics is generated based at least in part on extracting an oxygen saturation level at a sample rate of less than three seconds.

6. The system of claim 1, wherein the apnea classification comprises a hypoxia-related condition.

7. The system of claim 1, wherein the sleep apnea profile comprises oximetry data of an identified sleep apnea diagnosis from a prior sleep apnea study.

8. A portable system, comprising:
a sensor;
a computing device that comprises a processor and a memory, the computing device being attached to a patient for a sleep apnea diagnosis; and
machine-readable instructions stored in the memory that, when executed by the processor, cause the computing device to at least:
generate pulse oximetry data by measuring pulse oximetry of a patient with the sensor at a sampling rate of less than three seconds;
identify a plurality of sleep apnea indicators from the pulse oximetry data and from patient information associated with the patient;
provide the plurality of sleep apnea indicators to a machine learning model trained for sleep apnea prediction; and
receive a sleep apnea classification from the machine learning model.

9. The portable system of claim 8, further comprising a display, and the machine-readable instructions, when executed by the processor, cause the computing device to at least:
render on the display the sleep apnea classification.

10. The portable system of claim 8, wherein the machine-readable instructions, when executed by the processor, cause the computing device to at least:
transmit the sleep apnea classification to a mobile phone device via a wireless communication channel.

11. The portable system of claim 10, wherein the mobile phone device is configured to display a sleep apnea gauge that comprises a plurality of sleep apnea classifications and a user interface indicator referencing one of the plurality of sleep apnea classifications.

12. The portable system of claim 10, wherein the mobile phone device is configured to display a level of hypoxia and a user interface indicator referencing one of the plurality of hypoxia related conditions.

13. The portable system of claim 8, wherein the machine learning model is executed on at least one of an edge computing device, a mobile phone device, or a server.

14. The portable system of claim 8, wherein the senor comprises a light detector and a light emitting diode.

15. A method, comprising:
generating, by a portable device that includes a sensor, pulse oximetry data by measuring pulse oximetry of a patient with the sensor at a sampling rate of less than three seconds;
identifying, by the portable device, a plurality of sleep apnea indicators from the pulse oximetry data and from patient information associated with the patient;
providing, by the portable device, the plurality of sleep apnea indicators to a machine learning model trained for sleep apnea prediction; and
receiving, by the portable device, a sleep apnea classification from the machine learning model.

16. The method of claim 15, further comprising:
displaying, by the portable device, the sleep apnea classification.

17. The method of claim 15, further comprising:
displaying, by the portable device, a severity level for the sleep apnea classification.

18. The method of claim 17, further comprising:
transmitting, by the portable device, the sleep apnea classification to a mobile phone device via a wireless communication channel.

19. The method of claim 18, wherein the mobile phone device is configured to display a level of hypoxia and a user interface indicator referencing one of the plurality of hypoxia related conditions.

20. The method of claim 18, wherein the mobile phone device is configured to display a sleep apnea gauge that comprises a plurality of sleep apnea classifications and a user interface indicator referencing one of the plurality of sleep apnea classifications.
